(19) United States
(12) Patent Application Publication
(10) Pub. No.: US 2022/0062614 A1
(43) Pub. Date: Mar. 3, 2022

(54) CATHETER SYSTEMS AND RELATED METHODS

(71) Applicant: University of Kentucky Research Foundation, Lexington, KY (US)

(72) Inventors: Thomas Pittman, Lexington, KY (US); William Dillen, Lexington, KY (US)

(21) Appl. No.: 17/416,023

(22) PCT Filed: Dec. 20, 2019
(86) PCT No.: PCT/US2019/067990

§ 371 Date: Jun. 18, 2021

Related U.S. Application Data
(60) Provisional application No. 62/783,021, filed on Dec. 20, 2018.

(51) Int. Cl. A61M 39/02; A61M 25/02

(52) U.S. Cl. CPC A61M 39/0247; A61M 25/02; A61M 2025/0246; A61M 2205/123; A61M 2202/0464; A61M 2039/0291; A61M 2039/0205; A61M 2039/025; A61M 2039/0261; A61M 2039/0273; A61M 2210/0693

(57) ABSTRACT

A catheter system includes abase plate having a bottom surface, an outer circumferential wall, a circular flange extending inwardly from the outer circumferential wall, and an inner ring that is spaced apart from the outer circumferential wall, extends upwardly from the bottom surface, and defines a central opening in the base plate. A catheter is placed within the central opening of the base plate. A cover is placed over and secured to the base plate. The cover has a closed upper portion defining an internal chamber of the cover and a lower portion defining an opening in the cover. The lower portion includes a skirt with a flange extending outwardly around a periphery of the skirt to engage the circular flange of the base plate and form a closed reservoir. Methods of accessing cerebrospinal fluid using the catheter systems are also provided.

### RELATED APPLICATIONS

This application claims priority from U.S. Provisional Application Ser. No. 62/783,021 filed Dec. 20, 2018, the entire disclosure of which is incorporated herein by this reference.

### TECHNICAL FIELD

The present invention relates to catheter systems and related methods. In particular, certain embodiments of the present invention relate to catheter systems that form a closed reservoir and that are useful as an intraventricular catheter system for accessing cerebrospinal fluid of a subject.

### BACKGROUND

To aspirate cerebrospinal fluid or to deliver drugs (e.g. chemotherapy) into the cerebrospinal fluid of a subject, surgeons routinely make use of an intraventricular catheter system known as the Ommaya reservoir or Ommaya system. Once placed, the Ommaya reservoir can be used to treat brain tumors, leukemia/lymphoma, or leptomeningeal disease by intrathecal drug administration. Moreover, in the palliative care of terminal cancer patients, an Ommaya reservoir may be placed to allow for the intracerebroventricular injection (ICV) of morphine.

The Ommaya reservoir itself consists of a minimal number of parts, namely a catheter that is placed in one lateral ventricle of a subject, a reservoir attached to the catheter and implanted under the scalp of the subject, and a stylet (e.g., a wire) that is used to guide the placement of the catheter. Despite consisting of such a minimal number of parts, however, the placement of the Ommaya system requires two operators and is generally regarded as cumbersome. In particular, to place the Ommaya reservoir, the patient is first positioned and the head is fixed to the bed where the patient's head is then registered with neuro-navigation. A planned incision and entry point on the skull is subsequently mapped on the scalp and the planned incision is drawn. The scalp is then shaved free of the hair and the head is prepped for surgery. During surgery, the scalp is incised, exposing the skull, and the planned site for a burr hole in the skull is checked again with neuro-navigation. The burr hole is then drilled in the skull and the dura is opened, with the catheter and accompanying stylet within its lumen subsequently being passed into the brain. The next portion of the surgery includes a navigated portion to ensure appropriate placement of the catheter and, once the catheter has been appropriately placed, the stylet is removed, leaving the catheter in place. At that time, cerebrospinal fluid (CSF) can be seen coming from the catheter, which reassures that it is in the appropriate position, but also consequently causes the ventricles in the brain of the subject to decrease in size as the CSF escapes. As such, the reservoir of the Ommaya reservoir must then be quickly attached to the catheter.

The attachment of the reservoir, however, is typically regarded as the most cumbersome and risky part of the placement procedure, as it requires two operators and excessive manipulation of the catheter. More specifically, in placing the reservoir, the catheter must first be grasped and cut to length. After appropriate sizing, the catheter is then placed over the male coupling/nipple on the underside of the reservoir. The catheter fits tightly over the coupling though, thus making the catheter difficult to place, which, in turn, is further complicated by the minimal space in which to work and the importance of avoiding excessive manipulation of the catheter. In this regard, if the catheter were to be pulled from the brain, it could jeopardize the subject and the placement of the system as some C SF has inevitably been lost at that point and the ventricles in the brain of the subject may have collapsed down. Moreover, in attaching the reservoir, it is also important to avoid pushing the catheter into the brain or ventricle any further during the process as the catheter could damage deep brain structures. Accordingly, a catheter system and related method that allows for a safer and more efficient installation of such a system into the brain of a subject would be both highly desirable and beneficial.

### SUMMARY

The present invention includes catheter systems and related methods. In particular, certain embodiments of the present invention include catheter systems that form a closed reservoir and that are useful as an intraventricular catheter system for accessing cerebrospinal fluid of a subject.

In one embodiment of the present invention, a catheter system is provided that includes a base plate and a cover that is configured to be placed over and secured to the base plate. The base plate has bottom surface and an outer circumferential wall that extends upwardly from the bottom surface of the base plate. A circular flange is also included in the base plate and extends inwardly from the outer circumferential wall. Further, the base plate includes an inner ring that is spaced apart from the outer circumferential wall and extends upwardly from the bottom surface such that the inner ring is generally positioned in the center of the base plate and defines a central opening in the base plate.

The cover of the catheter system is configured to be placed over and secured to the base plate. The cover has a closed upper portion that defines an internal chamber of the cover and a lower portion that defines an opening in the cover. The lower portion also includes a skirt with a flange that extends outwardly around a periphery of the skirt to thereby engage the circular flange of the base plate. In this way, upon placement of the cover on the base plate, the cover and base plate form a closed reservoir or, in other words, a sealed reservoir system.

Further included in the catheter system is a catheter that defines an interior lumen and is configured to be placed within the central opening of the base plate. The catheter system further makes use of and includes a rigid stylet that is inserted within the interior lumen of the catheter and includes a hollow interior to allow cerebrospinal fluid to pass through the stylets. The stylet further includes a groove at the distal end of the stylet to provide tactile feedback to a surgeon inserting the catheter system and to thereby identify when the distal end of the stylet is approaching the central opening of the base plate. At that point, the distal end of the stylet can then be separated from the remainder of the stylet at the point of the groove. Upon securing the cover to the base plate, the stylet then provides a further rigid structure that more firmly secures the catheter in the inner ring of the base plate and helps to maintain the patency of the inner lumen of the catheter.

The catheter systems described herein can also be used in various methods of accessing cerebrospinal fluid of a subject. In one implementation of such a method of accessing cerebrospinal fluid, a burr hole is first drilled and placed in the dura of a particular subject. A base plate of an exemplary catheter system is then positioned over the burr hole and secured to the subject. The catheter of the system is subsequently inserted through the central opening of the base plate and into a ventricle of the brain of the subject. A portion of the stylet can then be removed from catheter and the cover placed onto the base plate. Once placed, the catheter system forms a closed reservoir that can then, in certain implementations, be punctured with a needle to access the cerebrospinal fluid so as to deliver therapeutic agents into the cerebrospinal fluid or remove an amount of the cerebrospinal fluid.

Further features and advantages of the present invention will become evident to those of ordinary skill in the art after a study of the description, figures, and non-limiting examples in this document.

### BRIEF DESCRIPTION OF THE DRAWINGS

FIG. 1 is a top view of a base plate for a catheter system made in accordance with the present invention;

FIG. 2 is cross-sectional side view of the base plate of FIG. 1;

FIG. 3 is a cross-sectional side view of a cover for a catheter system made in accordance with the present invention and configured to be placed over and secured to the base plate of FIG. 1;

FIG. 4 is a cross-sectional side view of another base plate for a catheter system made in accordance with the present invention;

FIG. 5 is a cross-sectional side view of another cover for a catheter system made in accordance with the present invention and configured to be placed over and secured to the base plate of FIG. 4;

FIGS. 6A-6C are side views of various stylets for a catheter system made in accordance with the present invention;

FIG. 7 is another side view of the base plate of FIGS. 1-2, and further showing a catheter and distal end of a stylet placed in the central opening of the base plate;

FIG. 8 is a perspective view of an assembled catheter system made in accordance with the present invention; and

FIG. 9 is a schematic diagram showing the use of a catheter system made in accordance with the present invention in accessing the cerebrospinal fluid of a subject.

### DESCRIPTION OF EXEMPLARY EMBODIMENTS

The present invention includes catheter systems and related methods. In particular, certain embodiments of the present invention include catheter systems that form a closed reservoir and that are useful as an intraventricular catheter system for accessing cerebrospinal fluid of a subject.

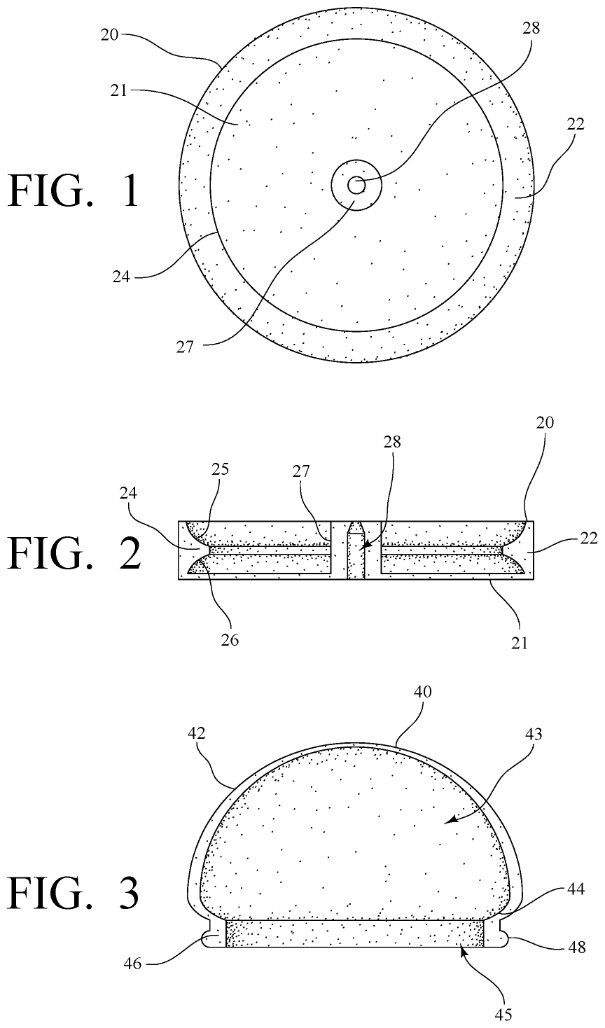

Referring first to FIGS. 1-3, in one exemplary embodiment of a catheter system made in accordance with the present invention, a catheter system is provided that includes a base plate 20 and a cover 40 that is configured to be placed over and secured to the base plate 20. The base plate 20 has a generally flat bottom surface 21 and an outer circumferential wall 22 that extends upward from the bottom surface 21 of the base plate 20. A circular flange 24 having a beveled upper surface 25 and a beveled lower surface 26 is also included in the base plate 20 and extends inwardly from the outer circumferential wall 22. Further, the base plate 20 also includes an inner ring 27 that extends upwardly from the bottom surface 21 and is spaced apart from the outer circumferential wall 22 such that the inner ring 27 is positioned in the center of the base plate 20 and defines a central opening 28 in the base plate 20.

With respect to the cover 40 of the catheter system, and as shown best in FIG. 3, the cover 40 has a dome-shaped and closed upper portion 42 that defines an internal chamber 43. The cover 40 also has a lower portion 44 that defines an opening 45 in the cover 40. The lower portion also includes a skirt 46 with a flange 48 extending outwardly around the periphery of the skirt 46. In this regard, as the cover 40 is placed onto the base plate 20 of the catheter system, the flange 48 extending around the skirt 46 engages the beveled surfaces 25, 26 of the circular flange 24 of the base plate 20 such that the cover 40 “snaps” onto the base plate 20 to thereby form a closed reservoir, as described in further detail below.

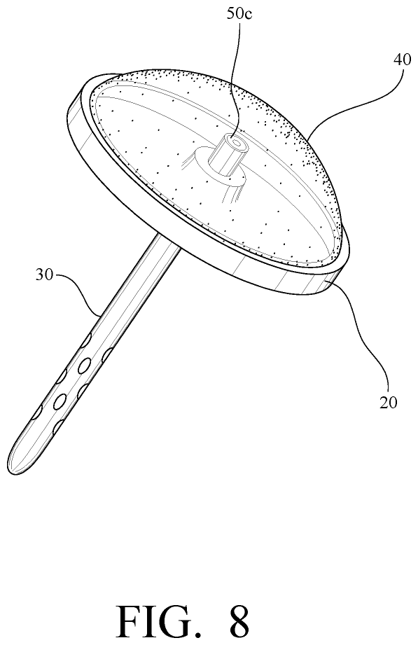

Referring now to FIGS. 7 and 8, and in addition to the base plate 20 and cover 40, the catheter system further includes and makes use of a catheter 30 that defines an interior lumen 32 and is configured to be placed within the central opening 28 of the base plate 20, as also described below. The catheter 30 is of the type and construction typically found in Ommaya reservoir systems, but will generally have a diameter equal to or slightly greater than the diameter of the central opening 28 such that placement of the catheter 30 within the central opening seals the catheter 30 against the central opening 28 and provides, in conjunction with the placement of the cover 40 on the base plate 20, a sealed reservoir system.

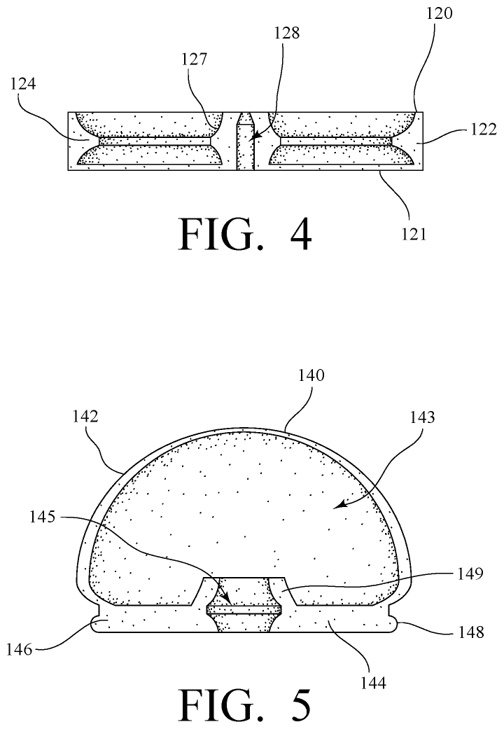

To further ensure a sealed system, and referring now to FIGS. 4-5, in another embodiment of the present invention, a catheter system is provided that, similar to the catheter system shown in FIGS. 1-3, includes a base plate 120 having a bottom surface 121, an outer circumferential wall 122, a circular flange 124 extending inwardly from the outer circumferential wall 122, and an inner ring 127 extending upwardly from the center of the base plate 120 and defining a central opening 128 in the base plate 120. The catheter system shown in FIGS. 4-5 also includes a dome-shaped cover 140 that is configured to be placed over the base plate 120, and that has a closed upper portion 142 defining a central chamber 143 and a lower portion 144 defining an opening 145 in the cover 140. The lower portion 144 further includes a skirt 146 with a flange 148 extending around the periphery of the skirt 146. Unlike the catheter system shown in FIGS. 1-3, however, the catheter system shown in FIGS. 4-5 and, more particularly, the lower portion 144 of the cover 140 further includes a bottom surface having a circular collar 149 that defines the opening 145 in the cover 140 and that extends upwardly towards and into the closed upper portion 142 of the cover 140. In this way, when the cover 140 is placed on the base plate 120 and a catheter is placed in the central opening 128 of the base plate 120, the circular collar 149 engages corresponding beveled surfaces on the inner ring 127 of the base plate 120 and causes the base plate 120 to crimp or otherwise push the inner ring 127 of the base plate against a placed catheter and further seal the system.

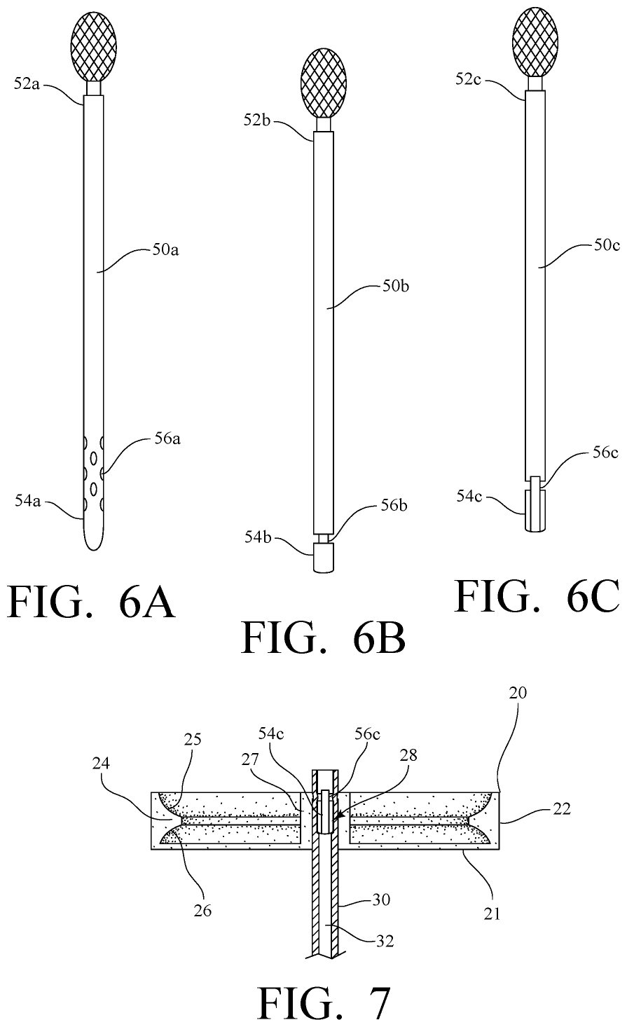

Regardless of the particular configuration of the base plates and covers included in the exemplary catheter systems of the present invention, and referring now to FIGS. 6A-6C, the catheter systems further include and make use of rigid stylets 50a, 50b, 50c having proximal ends 52a, 52b, 52c and distal ends 54a, 54b, 54c, and which assist in the insertion and placement of the catheters used in accordance with the present invention. Unlike prior stylets utilized in prior catheter systems, the stylets 50a, 50b, 50c define and include a hollow interior to allow cerebrospinal fluid to pass through the stylets 50a, 50b, 50c upon insertion of one of the stylets 50a, 50b, 50c into a catheter. Further, unlike prior stylets, each of the stylets 50a, 50b, 50c further include grooves 56a, 56b, 56c at the distal ends 54a, 54b, 54c of the stylets 50a, 50b, 50c. In this way, and as perhaps shown best in FIGS. 7 and 8, upon insertion of a catheter 30 into the brain of a subject with a particular stylet 50c placed in the lumen 32 of the catheter 30, as the stylet 50c is withdrawn from the catheter 30, the groove 56c provides tactile feedback to the surgeon inserting the catheter system to thereby identify when the distal end 54c of the stylet 50c is approaching or at the central opening 28 defined by the inner ring 27 of the base plate 20. At that point, the distal end 54c of the stylet 50b can then be broken off or otherwise separated from the remainder of the stylet 50b at the point of the groove 56c such that the distal end 54c of the stylet 50c remains within the inner ring 27. Upon the snapping on of a cover onto the base plate 20, such as the cover 40 or cover 140 shown in FIG. 3 or 5, respectively, the distal portion 54c of the stylet 50c provides a rigid structure that more firmly secures the catheter 30 in the inner ring 27 while maintaining the patency of the inner lumen of the catheter 30.

Of course, the grooves 56a, 56b, 56c included in the stylets 50a, 50b, 50c as well as the distal ends 54a, 54b, 54c of the stylets 50a, 50b, 50c can be provided in a number of shapes and configurations to provide the above-described tactile feedback to a surgeon and/or to provide a sufficient structure to secure the catheter in the inner ring 127 of the base plate. For example, in the embodiment shown in FIG. 6A, the stylet 50a is cannulated such that the grooves 56a are in the form a plurality of fenestrations that are in communication with the hollow interior of the stylet 50a. As another example, in the embodiment shown in FIG. 6C, the distal end 54c of the stylet 50c is plus-shaped to provide a structure capable of sufficiently securing the stylet 50c in a catheter, while allowing space for the cerebrospinal fluid to pass through the catheter.

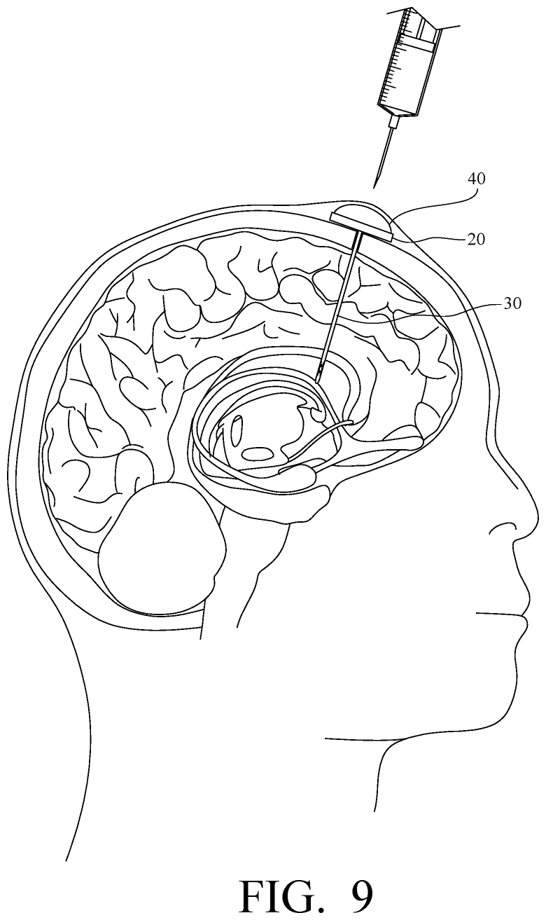

Each of the catheter systems described herein can also be used in methods of accessing cerebrospinal fluid of a subject, as shown, for example, in FIG. 9. In one exemplary implementation of a method of accessing cerebrospinal fluid of a subject in accordance with the present invention, a burr hole is first drilled and placed in the dura of particular subject, and a base plate of the catheter systems described herein is subsequently positioned over the burr hole and secured to the subject. Once the base plate is in place, the catheter and stylet of an exemplary system is then passed through the central opening of the base plate and into the brain until the target of the catheter (e.g., a ventricle) is reached. As described above, the stylet can then be pulled back until a tactile pop or other such signal is felt, indicating that a groove on the distal end of the stylet was at the level of the crimping mechanism/inner ring of the base plate. The distal end of the stylet is then broken off from the proximal portion of the stylet, leaving the distal end within the catheter. The catheter is then cut to length and the reservoir is snapped onto the base plate to seal the system and, in certain implementations, cause the base plate to crimp the catheter against the distal end of the stylet remaining in the lumen. Once placed, the catheter system and, more specifically, the closed reservoir formed by the catheter system, can then be punctured with a needle and cerebrospinal fluid can be accessed by drawing it from the system.

In some implementations, by making use of a catheter system described herein, the need for a second operator is eliminated as the entire process can be performed by a single person. In some implementations, the use of an exemplary catheter system also limits the manipulation of the catheter, as the catheter does not need to be moved after being initially placed in position. Further, in some implementations, the outer circumferential wall included in an exemplary base plate can also define an outlet or opening (not shown) for connecting an additional distal catheter such that the catheter systems of the present invention can be utilized for ventriculoperitoneal (VP) shunts as well, where a distal catheter is attached to the formed closed reservoir and is tunneled to the subject's abdomen. In this regard, in yet further implementations, the cover of an exemplary catheter system can also be attached to a connection such that the catheter system serves as a proximal portion of a shunt, while the connection positioned on the cover attaches the catheter system to distal shunt components. In this way, in such implementations, the catheter systems of the present invention can function similar to a Rickman Reservoir, but yet allow the catheter of the system to be placed within the ventricle of a subject and then secured in place within the cover.

Finally, each of the components of catheter systems described herein, with the possible exception of the stylets, are generally comprised of a pliant material, such as pliable plastic or silicone materials that known to those to skilled in the art for use in surgical applications, and which are sufficiently pliable so as not cause pressure issues with tissues overlying the materials, but yet are firm enough to allow the cover of an exemplary system to maintain their shape upon implantation under the scalp of s subject. In this regard, with respect to the subjects described herein, as used herein, the term “subject” includes both human and animal subjects. Thus, veterinary therapeutic uses are also provided in accordance with the present invention.

One of ordinary skill in the art will recognize that additional embodiments are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A catheter system, comprising:
a base plate having a bottom surface, an outer circumferential wall extending upwardly from the bottom surface, a circular flange extending inwardly from the outer circumferential wall, and an inner ring spaced apart from the outer circumferential wall and extending upwardly from the bottom surface, the inner ring defining a central opening in the base plate;
a catheter defining a lumen and configured to be placed within the central opening; and
a cover configured to be placed over and secured to the base plate, the cover having a closed upper portion defining an internal chamber of the cover and a lower portion defining an opening in the cover, the lower portion including a skirt with a flange extending outwardly around a periphery of the skirt for engaging the circular flange of the base plate such that, upon placement of the cover on the base plate, the cover and base plate form a closed reservoir.

2. The catheter system of claim 1, wherein the lower portion of the cover includes a bottom surface having a circular collar for surrounding the inner ring of the base plate.

3. The catheter system of claim 2, wherein the circular collar defines the opening in the cover and extends upwardly towards the closed upper portion of the cover.

4. The catheter system of claim 1, wherein the cover is dome-shaped.

5. The catheter system of claim 1, wherein the circular flange of the base plate includes a beveled upper surface and a beveled lower surface.

6. The catheter system of claim 1, further comprising a stylet for placement in the lumen of the catheter, the stylet including a proximal end and a distal end.

7. The catheter system of claim 6, wherein the distal end of the stylet includes a groove on an outer surface of the stylet.

8. The catheter system of claim 6, wherein the stylet defines a hollow interior.

9. The catheter system of claim 1, wherein the outer circumferential wall of the base plate further defines an outlet for connecting to a distal catheter.

10. The catheter system of claim 1, wherein the base plate and the cover are comprised of a pliant material.

11. A catheter system, comprising:
a base plate having a bottom surface, an outer circumferential wall extending upwardly from the bottom surface, a circular flange extending inwardly from the outer circumferential wall, and an inner ring spaced apart from the outer circumferential wall and extending upwardly from the bottom surface, the inner ring defining a central opening in the base plate;
a catheter defining a lumen and configured to be placed within the central opening;
a stylet for placement in the lumen of the catheter, the stylet including a proximal end and a distal end; and
a cover configured to be placed over and secured to the base plate, the cover having a closed upper portion defining an internal chamber of the cover and a lower portion defining an opening in the cover, the lower portion including a skirt with a flange extending outwardly around a periphery of the skirt for engaging the circular flange of the base plate such that, upon placement of the cover on the base plate, the cover and base plate form a closed reservoir.

12. The catheter system of claim 11, wherein the distal end of the stylet includes a groove on an outer surface of the stylet.

13. The catheter system of claim 11, wherein the stylet defines a hollow interior.

14. A method of accessing the cerebrospinal fluid of a subject, comprising:
providing a catheter system including a base plate having a bottom surface, an outer circumferential wall extending upwardly from the bottom surface, a circular flange extending inwardly from the outer circumferential wall, and an inner ring spaced apart from the outer circumferential wall and extending upwardly from the bottom surface, the inner ring defining a central opening in the base plate,
a catheter defining a lumen,
a stylet including a proximal end and a distal end, and
a cover having a closed upper portion defining an internal chamber of the cover and a lower portion defining an opening in the cover, the lower portion including a skirt with a flange extending outwardly around a periphery of the skirt;
placing a burr hole through the dura of a subject;
securing the base plate over the burr hole;
placing the catheter with the stylet placed in the lumen of the catheter through the central opening of the base plate and into a ventricle of a brain of the subject;
removing at least a portion of the stylet from the catheter; and
securing the cover to the base plate.

15. The method of claim 14, wherein removing at least a portion of the stylet comprises removing the proximal portion of the stylet from the catheter.

16. The method of claim 15, further comprising separating the proximal portion from the distal portion of the stylet such that the distal portion of the stylet remains within the lumen of catheter adjacent to the central opening of the inner ring of the base plate.

17. The method of claim 14, wherein the distal end of the stylet includes a groove on an outer surface of the stylet.

18. The method of claim 14, wherein the stylet defines a hollow interior.

19. The method of claim 14, further comprising, after securing the cover to the base plate, a step of piercing the cover to access the cerebrospinal fluid of the subject.

20. The method of claim 19, wherein piercing the cover comprises piercing the cover with a needle.
